(19) United States
(12) Patent Application Publication
(10) Pub. No.: US 2021/0161604 A1
(43) Pub. Date: Jun. 3, 2021

(54) SYSTEMS AND METHODS OF NAVIGATION FOR ROBOTIC COLONOSCOPY

(71) Applicant: Bnaiahu LEVIN, Ness Ziona (IL)

(72) Inventor: Bnaiahu LEVIN, Ness Ziona (IL)

(21) Appl. No.: 17/257,470

(22) PCT Filed: Jul. 15, 2019
(86) PCT No.: PCT/IL2019/050793

§ 371 Date: Dec. 31, 2020

Related U.S. Application Data
(60) Provisional application No. 62/699,185, filed on Jul. 17, 2018.

(51) Int. Cl. A61B 34/20; A61B 1/31; A61B 1/00; G06T 7/13; G06T 7/12; G06T 7/168; G16H 30/40; G16H 20/40

(52) U.S. Cl. CPC A61B 34/20; A61B 1/31; A61B 1/00149; G06T 7/13; G06T 7/12; G06T 7/168; G06T 2207/30028; G16H 20/40; A61B 2034/2055; A61B 2034/2065; G06T 2207/10068; G06T 2207/20048; G16H 30/40

(57) ABSTRACT

A computerized system and method of endoscope navigation is provided that includes receiving an endoscope image from the endoscope in a body lumen, determining from the endoscope image a proximate wall of the body lumen, determining a movement vector directing away from the proximate wall, and applying a mechanical motion to the endoscope, according to the movement vector, to move the endoscope away from the proximate wall.

### FIELD OF THE INVENTION

The present invention is directed to systems and methods for robotically assisted medical procedures, in particular for navigation of medical instruments for diagnosis and surgery.

### BACKGROUND

A colonoscope typically includes a light source and an image capture device and is externally steerable through the colon. Colonoscopy is frequently performed to screen for asymptomatic cancers at an early stage or to find and to remove precancerous polyps. Colonoscopy may also be performed to diagnose rectal bleeding or changes in bowel habits and inflammatory bowel disease. Although the procedure is common, safe navigation of a colonoscope through the colon can be difficult due to the colon's distensible and highly mobile nature. Colonoscopy complications that can occur include colon perforation, hemorrhage, or severe abdominal pain. Robotic control of colonoscope movement is being developed to provide greater precision and speed for the procedure; however, robotic control must also contend with the varying, dynamic anatomy of the colon.

Prevalent manual and automatic visual methods for navigating the colonoscope rely on directing the colonoscope towards dark regions of images acquired by the colonoscope. Khan and Gillies (“Vision based navigation for an endoscope”, Image and Vision Computing, 14 (1996), pp. 763-T72) describe determining and visually presenting the colon space, for manual and/or automatic navigation of the colon. The method includes directing the colonoscope towards a dark region of the lumen, according to the colonoscope image. The method also includes determining distances between muscle contours of the colon. Khan and Gillies note that a complete colon representation is necessary for guiding the endoscope around the bends when the view of the lumen is lost and muscle contours are not visible, or when the colon includes pockets or unforeseen obstacles.

U.S. Patent Publication 2003/0167007 to Belson is directed to a method of colonoscopy whereby a spectroscopy device is attached to the tip of the colonoscope to create a three dimensional map of the colon for use by an automated method of advancing the colonoscope. U.S. Pat. No. 8,795,157 to Yaron and Frenkel is directed to a method for advancing a colonoscope by acquiring a stereoscopic image pair of a region of the interior of a colon, identifying a plurality of topographical features on an inner wall of the colon, determining the depth of each topographical feature, determining a radius of curvature of the colon, and advancing the colonoscope according to the direction of the topographical feature with greatest depth, according to the radius of curvature.

U.S. Pat. No. 8,514,218 to Hong and Paladini is directed to navigating a colonoscope according to a depth image captured by an angular fish eye lens. In such a lens, the resolution is approximately equal across the whole image. The depth image is generated according to a ray casting volume rendering scheme. In the depth image, the gray level is proportional to the distance from the camera to the colon surface, the brighter region corresponding to the colon lumen which is far away from the current camera location, called the target region.

Similarly, U.S. Patent Publication 2003/0152897 to Geiger is directed to navigating a colonoscope by ray-casting, whereby for every pixel of an acquired colon image, a ray is cast and its intersection with an organ wall is calculated, to determine a longest ray. The colonoscope is then navigated in the direction of the longest ray.

The aforementioned methods do not overcome a variety of difficulties of colonoscope navigation that stem from the dynamic anatomy of the colon, as well as the highly varied surface structure. The colon may include dark pockets, including disease related distortions, such as diverticulitis. The colon image may also include image artifacts, such as fluids or bubbles on the lens. When the endoscope tip is close to the colon wall, the image may display ‘red-out’ or ‘wall view’. Camera movement may also cause motion blur artifacts. These complicating factors and artifacts have impeded successful implementation of automated navigation.

### SUMMARY

Embodiments of the present invention provide systems and methods for navigating an endoscope during colonoscopy. In some embodiments, a method of endoscope navigation includes: receiving an endoscope image from an endoscope in a body lumen; determining a wall section of the endoscope image representing a proximate wall of the body lumen; determining a movement vector directing away from the wall section of the image; and applying a mechanical motion to the endoscope, according to the movement vector, to move the endoscope away from the proximate wall. In further embodiments, the endoscope may be a colonoscope. Determining the wall section of the image may include determining a blurred portion of the endoscope image, and determining the blurred portion of the endoscope may include: generating by edge detection an edge-rendered mapping from the endoscope image, dividing the edge-rendered mapping into sectors, determining a variance of pixel intensity for each sector, and determining the blurred portion as a subset of the sectors having variances less than a threshold value.

In further embodiments the edge detection may be performed by applying a 3×3 Laplacian operator to the endoscope image to generate a second order derivative mapping.

The threshold value may be a variance less than a preset percentile of the variances of all the image subsections. Alternatively, the threshold value may be a preset variance value. The subset of the sectors having variances less than a threshold is a “blurred subset”, and determining the vector directing away from the wall section may include determining coordinates in the endoscope image of a center of gravity (COG) of the blurred subset. In some embodiments, the movement vector may be determined as a displacement from the coordinates of the COG towards a point in the endoscope image representing a current orientation of the endoscope tip. In alternative embodiments, the movement vector may be determined as a weighted average of a displacement from the coordinates of the COG towards a point in the endoscope image representing a current orientation of the endoscope tip, and of a displacement to one or more additional targets, include at least one of a dark region target and a previous endoscope image target.

In further embodiments, a system for endoscope navigation may include a processor and a memory with computer-readable instructions that when executed cause the processor to perform steps of: receiving an endoscope image from an endoscope in a body lumen; determining a wall section of the endoscope image representing a proximate wall of the body lumen; determining a movement vector directing away from the wall section of the image; and applying a mechanical motion to the endoscope, according to the movement vector, to move the endoscope away from the proximate wall.

The present invention will be more fully understood from the following detailed description of embodiments thereof.

### BRIEF DESCRIPTION OF DRAWINGS

In the following detailed description of various embodiments, reference is made to the following drawings that form a part thereof, and in which are shown by way of illustration specific embodiments by which the invention may be practiced, wherein:

FIG. 1 is a block diagram of a system for endoscope navigation, in accordance with an embodiment of the present invention;

FIG. 2 shows an endoscope image acquired during endoscope navigation, in accordance with an embodiment of the present invention;

FIG. 3 shows an edge-rendered mapping of the endoscope image, in accordance with an embodiment of the present invention;

FIG. 4 shows the edge-rendered mapping divided into sectors, in accordance with an embodiment of the present invention;

FIG. 5 shows a table of variances of pixel intensities of the sectors of the edge-rendered mapping, in accordance with an embodiment of the present invention;

FIG. 6 shows a blurred subset of the sectors, indicating proximity of a wall of a body lumen, in accordance with an embodiment of the present invention;

FIG. 7 shows vectors of several navigation targets, which are added to create a final target, during a process of endoscope navigation, in accordance with an embodiment of the present invention; and

FIG. 8 is a schematic, flow diagram of a process for endoscope navigation, according to an embodiment of the present invention.

### DETAILED DESCRIPTION OF THE INVENTION

In the following detailed description of various embodiments, it is understood that other embodiments may be utilized and structural changes may be made without departing from the scope of the present invention.

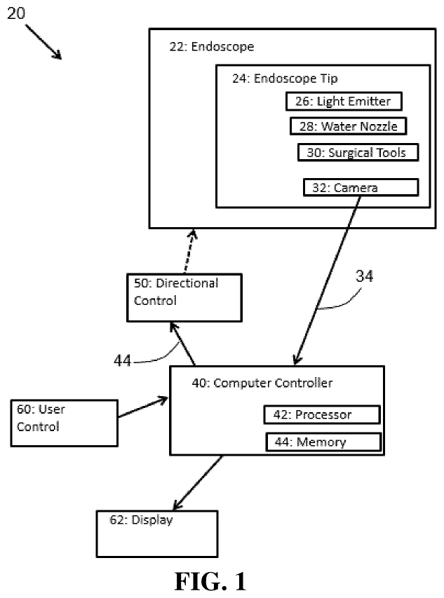

FIG. 1 is a block diagram of a system 20 for endoscope navigation, in accordance with an embodiment of the present invention. System 20 includes an endoscope 22 designed to be steered through a body lumen. In some embodiments is a colonoscope, and the body lumen is a patient's colon. Situated at the endoscope tip 24 may be several devices, such as a light emitter 26, a water nozzle 28, or other surgical instruments 30 (such as cautery probes, cold or hot forceps, snares, injection needles, suction, nets, or endoloops). The endoscope tip 24 also has a camera 32, which typically sends images as video frames to a computer controller 40. As described further hereinbelow, the lens system of the camera 32 is designed to provide a range of focus that is typically not less than 3 mm, and the steering of the endoscope is guided according to the range of focus.

The computer controller 40 typically includes a processor 42 and a memory 44, the memory including instructions for image processing described further hereinbelow. The computer controller may also be configured to receive input from a user control 60, which may, for example be steering instructions for controlling the endoscope 22. Alternatively or additionally, the computer controller 40 may be configured to control automated navigation of the endoscope 22 at least for a portion of the process of inserting and removing the endoscope from the. The computer controller 40 may also send navigation instructions 44 to a mechanical directional controller 50, for controlling a 3D position (i.e., x, y, and z axes) of the endoscope tip 24. The directional controller 50 typically has motors that operate cables of the endoscope 22 to move the endoscope tip 24.

The computer controller 40 may also present video from the camera 32 on a user display 62. Additional objects rendered by the computer controller 40, such as a navigation target may also be presented on the user display 62.

The endoscope tip may include additional navigation sensors, such as distance sensors and remote tracking sensors, not shown. The navigation methods provided by the present invention reduce the need for such additional sensors. However, measurements by such navigation sensors may also be applied to complement and to confirm the navigation process described hereinbelow.

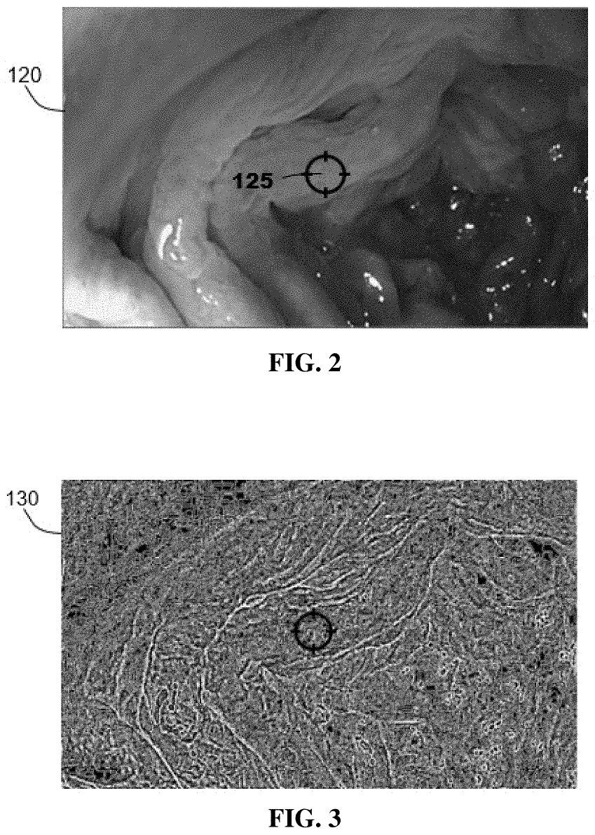

FIG. 2 shows an endoscope image 120 acquired during endoscope navigation, in accordance with an embodiment of the present invention. The computer controller 40 receives the endoscope image 120 from the camera 32, typically as a frame of a video transmission. Typically, the endoscope image is calibrated to indicate the orientation of the endoscope tip, such that the tip is oriented towards an image center 125. Alternatively, a current orientation of the endoscope tip may be indicated by other preset points of the image. The endoscope image 120 shown in the figure is an image of a colon, taken during a colonoscopy procedure.

Upon receiving the endoscope image 120, the computer controller 40 determines whether features of the image indicate that the endoscope tip is closer than a preset threshold to a wall of the body lumen. In an embodiment of the present invention, the computer controller processes the image to determine an out-of-focus (i.e., blurry) region, indicative of a region that is closer to the camera than the minimum focal length of the camera lens. Alternative, other methods of analyzing pixels of the image to determine wall proximity may be incorporated. For example, pixel saturation may be employed, as saturation may be indicative of high reflection very close to the endoscope light.

A blurry region may be categorized according to a method described in Pech-Pacheco, et al., “Diatom autofocusing in brightfield microscopy: a comparative study” (Proceedings 15th International Conference on Pattern Recognition, IEEE, Sept. 2000). An edge-rendered mapping is first generated by an edge detection operation. The variance of the pixel intensity in the mapping is then calculated. A high variance is indicative of good focus, while a low variance is indicative of poor focus.

An edge-rendered mapping of an image may be calculated by several methods known in the art, such as by convolution of the image with a discrete Laplace operator mask. A common mask for edge detection has the form:

$$[ \begin{matrix}0 & 1 & 0 \\ 1 & - 4 & 1 \\ 0 & 1 & 0\end{matrix} ] \qquad ( 1 )$$

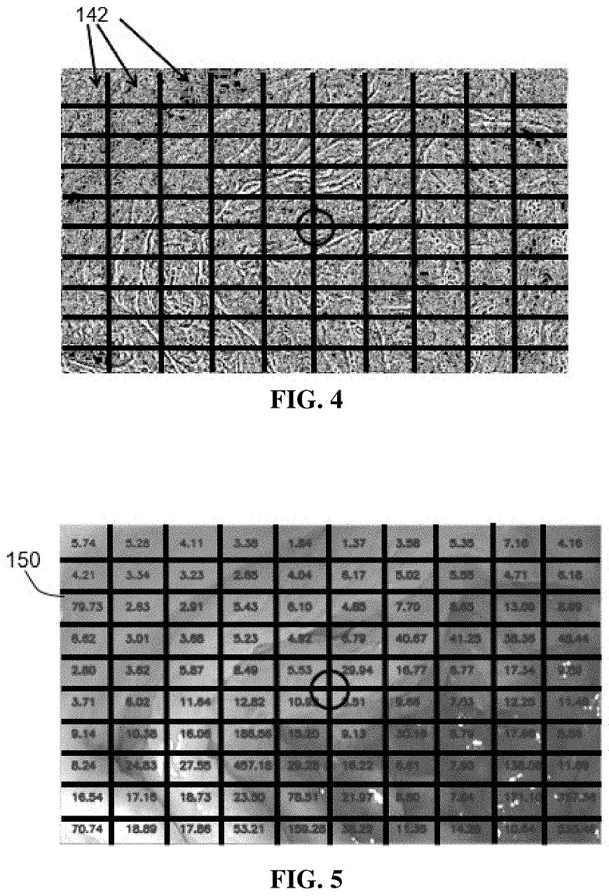

FIG. 3 shows an edge-rendered mapping 130, generated by detecting edges in the endoscope image, in accordance with an embodiment of the present invention. In order to differentiate between levels of blurriness in regions of the endoscope image, the edge-rendered mapping 130 is divided into sectors 142, as shown in FIG. 4. A variance of pixel intensities within each sector of the edge-rendered mapping is then calculated. FIG. 5 shows a table of variances 150 for each sector, in accordance with an embodiment of the present invention. The table is superimposed on the original endoscope image 120. The size of sectors for a given mapping may be varied, depending on factors such as camera resolution and quality. A typical sector size for a given mapping may be, for example 10×10 pixels.

After the variances are calculated for each sector of the endoscope image, the sectors may be differentiated between blurry sectors and focused sectors. In some embodiments, a preset threshold of sector variance is set to distinguish between blurry and non-blurry sectors. Alternatively, a percentile of the overall range of variances can be used to distinguish blurry from non-blurry sectors.

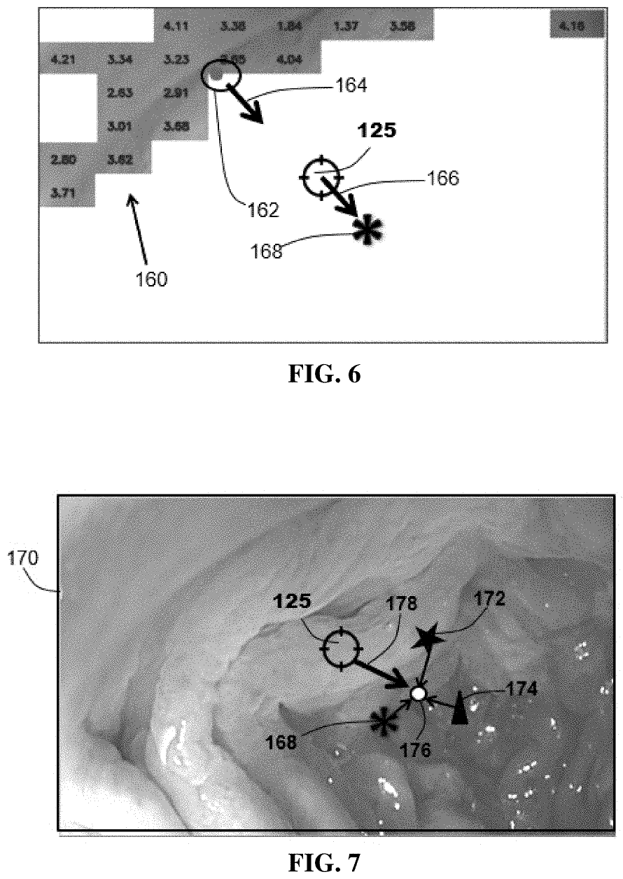

FIG. 6 shows a blurred subset 160 of the sectors, which have been distinguished as described above from the focused sectors. The blurriness of the blurred sectors is caused by the distance between a wall of the lumen (e.g., a wall of the colon) and the endoscope tip being less than the minimum field depth of the camera lens. Consequently the blurred subset represents a proximate wall of the lumen.

From the blurred subset 160 of the sectors, the computer controller calculates a direction for navigating the endoscope tip away from the proximate wall. The direction is determined as a vector extending from a “center of mass” or “center of gravity” (COG) of the blurred subset 160 of sectors towards a preset or interactively set point of the endoscope image, such as the center 125.

The variance is inversely proportional to a measure of blurriness; consequently, the COG, i.e., a “center of blurriness”, may be calculated by assigning to each sector an inverse value of the variance (e.g., a constant divided by the variance). The inverse variance value is used to represent mass for the COG calculation. Other inverse variance indices may also be used. In one embodiment, by way of example, the two-dimensional coordinates of the COG are calculated from a standard COG formula, as follows:

$$COG = \frac{\sum \frac{{r}_{i}}{{v}_{i}}}{\sum \frac{1}{{v}_{i}}} \qquad ( 2 )$$

where vᵢ is the variance of sector i, and rᵢ represents the coordinates of sector i, which may be, for example, coordinates of a central pixel of the sector, as measured from an arbitrary point, such as a corner or center point of the image. The summations in the formula for COG are summations over all sectors (i.e., i=1 to n).

The COG of the inverse variance of blurry sectors is indicated as point 162 in the figure.

The computer controller calculates a movement vector 164 starting from point 162 and extending in the direction of a point indicating a current orientation of the endoscope image 120, such as the center 125. The movement vector 164 indicates a directional motion which the computer controller then directs the direction controller 50 to apply to the endoscope.

In further embodiments, the origin of the movement vector 164 is transformed to the image center 125 (or other indicative orientation point), to generate a transformed vector 166. The transformed vector indicates a navigation target 168. FIG. 7 shows additional navigation targets, which may be aggregated with the navigation target 168 to create an aggregated final target 176. The additional navigation targets may be, for example, a previously calculated navigation target 172, calculated from a prior image, and a dark region target 174, calculated by identifying the center of the darkest region of the image, by methods of image processing known in the art. In one embodiment, the weights assigned to each target are preset. For example, the final target may be calculated by according an averaging weight of 50% to the previous target, 30% to the new target, and 20% to the dark region target.

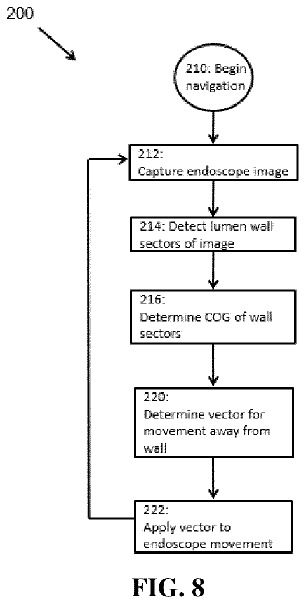

FIG. 8 is a schematic, flow diagram of a process 200 for endoscope navigation, according to an embodiment of the present invention. At an initial step 210, the navigation begins, with the insertion of the endoscope into the body lumen, for example, by beginning a colonoscopy procedure. An iterative process of computer-assisted or computer-controlled navigation then begins at a step 212, with the capture of the first endoscope image, typically a frame of an endoscope video.

At a step 214, pixels of the image are analyzed by computer methods to determine whether there is an indication of a proximate lumen wall in the image. The analysis may include methods described above for determining a section of saturated pixels, or alternatively or additionally a blurred area of the image, indicating that a wall of the lumen is less than the minimum focus length of the endoscope camera.

As described above, in some embodiments, the process of determining the blurred area includes detecting edges in the image by edge detection methods such as applying a Laplace operator to the pixels of the image to generate an edge-rendered mapping. Other methods of edge detection may also be employed to generate the edge-rendered mapping. After the edge-rendered mapping is generated, the mapping may be divided into sectors and the variance of pixel intensity in each sector calculated to generate an indication of blurriness, thereby indicating that the blurred subset of sectors is a wall section of the image.

At a step 216, the computer controller calculates a center of gravity (COG), which is a center of weighted values of the subset of pixel sectors that is determined to represent a proximate wall. When the proximate wall is determined by the variance of pixel intensity in the edge-rendered mapping, the weighted center may be calculated from inverse values of the variances.

At a step 220, the computer controller then calculates a vector extending from the COG to the center of the endoscope image to turn the endoscope tip away from the proximate wall. In further embodiments, the vector may also be calculated as a weighted average of multiple targets, which may include a prior vector target as well as a dark region target. At a step 222, the computer controller sends a signal indicative of the calculated vector to the mechanical directional controller of the endoscope to navigate the endoscope tip away from the wall, and then waits to receive a new image, that is, the process returns to step 212. The mechanical directional controller is pre-calibrated to convert the signal into an endoscope tip motion within a safe working range of operation.

It is to be understood that the embodiments described hereinabove are cited by way of example, and that the present invention is not limited to what has been particularly shown and described hereinabove. The scope of the present invention includes variations and modifications thereof which would occur to persons skilled in the art upon reading the foregoing description and which are not disclosed in the prior art. Computer processing elements described may be distributed processing elements, implemented over wired and/or wireless networks. Such computing systems may furthermore be implemented by multiple alternative and/or cooperative configurations, such as a data center server or a cloud configuration of processers and data repositories. Processing elements of the system may be implemented in digital electronic circuitry, or in computer hardware, firmware, software, or in combinations thereof. Such elements can be implemented as a computer program product, tangibly embodied in an information carrier, such as a non-transient, machine-readable storage device, for execution by, or to control the operation of, data processing apparatus, such as a programmable processor, computer, or deployed to be executed on multiple computers at one site or distributed across multiple sites. Memory storage may also include multiple distributed memory units, including one or more types of storage media.

Communications between systems and devices described above are assumed to be performed by software modules and hardware devices known in the art. Processing elements and memory storage, such as databases, may be implemented so as to include security features, such as authentication processes known in the art.

Method steps associated with the system and process can be rearranged and/or one or more such steps can be omitted to achieve the same, or similar, results to those described herein.

1. A method of endoscope navigation comprising:
receiving an endoscope image from an endoscope in a body lumen;
determining a wall section of the endoscope image representing a proximate wall of the body lumen, as a blurred section of the endoscope image;
determining a movement vector as a displacement from the blurred section; and
applying a mechanical motion to the endoscope, according to the movement vector, to move the endoscope away from the proximate wall.

2. The method of claim 1, wherein the endoscope is a colonoscope and the body lumen is a colon.

3. (canceled)

4. The method of claim 1, wherein determining the blurred portion of the endoscope image comprises: generating by edge detection an edge-rendered mapping from the endoscope image, dividing the edge-rendered mapping into sectors, determining a variance of pixel intensity for each sector, and determining the blurred portion as a subset of the sectors having variances less than a threshold value.

5. The method of claim 4, wherein the edge detection is performed by applying a 3×3 Laplacian operator to the endoscope image to generate a second order derivative mapping.

6. The method of claim 4, wherein the threshold value is a variance less than a preset percentile of the variances of all the image subsections.

7. The method of claim 4, wherein the threshold value is a preset variance value.

8. The method of claim 4, wherein the subset of the sectors having variances less than a threshold is a “blurred subset”, and wherein determining the movement vector directing away from the wall section comprises determining coordinates in the endoscope image of a center of gravity (COG) of the blurred subset.

9. The method of claim 8, wherein the movement vector is determined as a displacement from the coordinates of the COG towards a point in the endoscope image representing a current orientation of the endoscope tip.

10. The method of claim 8, wherein the movement vector is determined as a weighted average of a displacement from the coordinates of the COG towards one or more target points, wherein the one or more target points include one or more of:
a point in the endoscope image representing a current orientation of the endoscope tip; a dark region target;
and a previous endoscope image target.

11. A system for endoscope navigation, comprising a processor and a non-transient memory with computer-readable instructions that when executed cause the processor to perform steps of:
receiving an endoscope image from an endoscope in a body lumen;
determining a wall section of the endoscope image representing a proximate wall of the body lumen, as a blurred section of the endoscope image;
determining a movement vector as a displacement from the blurred section; and
applying a mechanical motion to the endoscope, according to the movement vector, to move the endoscope away from the proximate wall.

12. The system of claim 11, wherein the endoscope is a colonoscope and the body lumen is a colon.

13. (canceled)

14. The system of claim 11, wherein determining the blurred portion of the endoscope image comprises: generating by edge detection an edge-rendered mapping from the endoscope image, dividing the edge-rendered mapping into sectors, determining a variance of pixel intensity for each sector, and determining the blurred portion as a subset of the sectors having variances less than a threshold value.

15. The system of claim 14, wherein the edge detection is performed by applying a 3×3 Laplacian operator to the endoscope image to generate a second order derivative mapping.

16. The system of claim 14, wherein the threshold value is a variance less than a preset percentile of the variances of all the image subsections.

17. The system of claim 14, wherein the threshold value is a preset variance value.

18. The system of claim 14, wherein the subset of the sectors having variances less than a threshold is a “blurred subset”, and wherein determining the movement vector comprises determining coordinates in the endoscope image of a center of gravity (COG) of the blurred subset.

19. The system of claim 18, wherein the movement vector is determined as a displacement from the coordinates of the COG towards a point in the endoscope image representing a current orientation of the endoscope tip.

20. The system of claim 18, wherein the movement vector is determined as a weighted average of a displacement from the coordinates of the COG towards one or more target points, wherein the one or more target points include one or more of: a point in the endoscope image representing a current orientation of the endoscope tip; a dark region target; and a previous endoscope image target.

21. A system for colonoscope navigation, comprising a processor and a non-transient memory with computer-readable instructions that when executed cause the processor to perform steps of:
receiving an colonoscope image from an colonoscope in a colon;
determining a wall section of the colonoscope image representing a proximate wall of the colon, as a blurred section of the colonoscope image;
determining a movement vector as a displacement from the blurred section; and
applying a mechanical motion to the colonoscope, according to the movement vector, to move the colonoscope away from the proximate wall.
